(19) United States
(12) Patent Application Publication
(10) Pub. No.: US 2014/0364704 A1
(43) Pub. Date: Dec. 11, 2014

(54) APPARATUS FOR DETECTING SURFACE MICROCIRCULATION OF ACUPOINT

(71) Applicant: National Taiwan University of Science and Technology, Taipei City (TW)

(72) Inventor: Hsin HSIU, TAIPEI CITY (TW)

(21) Appl. No.: 14/298,979

(22) Filed: Jun. 9, 2014

(30) Foreign Application Priority Data
Jun. 11, 2013 (TW) 102120759

(51) Int. Cl. A61B 5/00

(52) U.S. Cl. CPC A61B 5/4854
USPC 600/301; 600/479

(57) ABSTRACT

An apparatus for detecting surface microcirculation of an acupoint includes an acupoint locator, a bio-optical blood flow detection device and a signal analysis processor. The acupoint locator includes an optical probe disposed therein and is configured to let the optical probe substantially align with and be in contact with a surface of the acupoint of a human subject. The bio-optical blood flow detection device is configured to capture microcirculation signals of the surface of the acupoint of the human subject by the optical probe. The signal analysis processor is configured to receive and analyze the microcirculation signals.

### RELATED APPLICATIONS

This application claims priority to Taiwan Application Serial Number 102120759, filed Jun. 11, 2013, which is herein incorporated by reference.

### BACKGROUND

1. Field of Invention

The present disclosure relates to an apparatus for detecting surface microcirculation of an acupoint.

2. Description of Related Art

Personal blood glucose detectors in the market primarily are to use finger prick blood test. Such invasive blood glucose detectors almost occupy all the market of the personal blood glucose detectors. However, a user may feel pain due to a needle, and wound is not easy to recover, and amount of bleeding may be not enough to test and thus the user requires to repeat the test, such that diabetics are often reluctant to use the invasive blood glucose detectors, which results in inadequate effective detection times and derogation of clinical value. Therefore, a non-invasive detection method for diabetics has great application significance in the development of home biomedical electronics and telemedicine. In addition, if an effective early detection method with low damage is provided, associated health care cost may be reduced and the market of the diabetes detection devices may be increased. However, there has not been any non-invasive detection device able to continuously monitor blood glucose, and to understand long variation of a relationship between blood glucose concentration and metabolism, and to feature accuracy, easy to use and low cost.

Optical measurement is non-invasive and exhibits high affinity, low damage and small volume, and thus biomedical optical measurement becomes an important trend in current biomedical measurements. Nevertheless, due to high attenuation and scattering in body tissue and individual differences, optical signals are not easy to analyze or interpret. Accordingly, there has not been non-invasive measurement techniques or devices able to be used in chronic disease detection and monitoring of disease progression.

Optical measurement techniques have been applied in evaluation of peripheral blood supply. For example, a microscopic imaging method may be used in evaluation of microcirculation, which is evaluated by counting erythrocytes; however, the microscopic imaging method may only be applied to thin tissues such as mesenteric artery, nail fold microcirculation, eye conjunctiva microcirculation and so on. In clinical practice, resolution of measurement technology of microcirculation still fails to effectively distinguish blood flow change due to various diseases progression. In one practical application, the optical measurement technique is applied to skin burn patients, which is used to monitor statuses of skin growth. However, the optical measurement technique cannot be applied in early detection of diseases. Therefore, there is a need for a detection device, which exhibits good resolution between patients with chronic diseases and normal individuals, to effectively apply in early detection of such diseases.

### SUMMARY

An aspect of the present disclosure provides an apparatus for detecting surface microcirculation of an acupoint. The apparatus includes an acupoint locator, a bio-optical blood flow detection device and a signal analysis processor. The acupoint locator includes an optical probe disposed therein and is configured to let the optical probe substantially align with and be in contact with a surface of the acupoint of a human subject. The bio-optical blood flow detection device is configured to capture microcirculation signals of the surface of the acupoint of the human subject by the optical probe, in which the bio-optical blood flow detection device is a laser Doppler flowmetry (LDF) or a photoplethysmography (PPG). The signal analysis processor is configured to receive and analyze the microcirculation signals.

According to one embodiment of the present disclosure, the apparatus further includes a reference locator and a spacing fixer. The reference locator is configured to be in contact with a surface of a joint of the human subject, and the joint is adjacent to the acupoint. Two opposite sides of the spacing fixer are respectively connected to the reference locator and the acupoint locator.

According to one embodiment of the present disclosure, the acupoint is taihsi acupoint or sanyinjiao acupoint, and the surface of the joint is internal ankle tip.

According to one embodiment of the present disclosure, the reference locator has an opening configured to substantially align with and accommodate the internal ankle tip.

According to one embodiment of the present disclosure, the bio-optical blood flow detection device is the laser Doppler flowmetry, and the signal analysis processor is configured to transform the microcirculation signals to total energy of a specific frequency band, and the specific frequency band is in a range of about 0.0095 Hz to about 0.021 Hz, about 0.021 Hz to about 0.052 Hz, about 0.052 Hz to about 0.145 Hz, about 0.145 Hz to about 0.6 Hz or about 0.6 Hz to about 1.6 Hz.

According to one embodiment of the present disclosure, the acupoint is taihsi acupoint, and the apparatus is configured for early detection of diabetes.

According to one embodiment of the present disclosure, the acupoint is sanyinjiao acupoint, and the apparatus is configured for early detection of polycystic ovary syndrome.

According to one embodiment of the present disclosure, the apparatus further includes an electrode patch and a physiological signal amplifier. The electrode patch is configured to paste on a surface of the human subject. The physiological signal amplifier is configured to capture and amplify electrocardiogram (ECG) signals through the electrode patch.

According to one embodiment of the present disclosure, the bio-optical blood flow detection device is the laser Doppler flowmetry, and the signal analysis processor is further configured to receive and analyze the electrocardiogram signals to obtain a foot delay time (FDT).

According to one embodiment of the present disclosure, the acupoint is hegu acupoint.

According to one embodiment of the present disclosure, the apparatus further includes an elastic element configured to position the acupoint locator.

### BRIEF DESCRIPTION OF THE DRAWINGS

The disclosure can be more fully understood by reading the following detailed description of the embodiment, with reference made to the accompanying drawings as follows:

FIG. 1 is a functional module diagram of an apparatus for detecting surface microcirculation of an acupoint according to one embodiment of the present disclosure.

FIG. 2 is a schematic diagram of an acupoint locator immobilized on hegu acupoint according to one embodiment of the present disclosure.

FIG. 3 is a functional module diagram of an apparatus for detecting surface microcirculation of an acupoint according to another embodiment of the present disclosure,

FIGS. 4A-4B respectively are top and cross-sectional views of an acupoint locator, a spacing fixer and a reference locator according to one embodiment of the present disclosure.

FIG. 4C is a schematic diagram of an acupoint locator immobilized on taihsi acupoint according to one embodiment of the present disclosure.

FIG. 5 is a functional module diagram of an apparatus for detecting surface microcirculation of an acupoint according to further embodiment of the present disclosure.

FIG. 6 is a relationship diagram of relative energy contribution of each frequency band according to Example 1 of the present disclosure.

FIG. 7 is a relationship diagram of foot delay time according to Example 1 of the present disclosure.

FIG. 8 is a relationship diagram of relative energy contribution of each frequency band according to Comparative Example 1 of the present disclosure.

FIG. 9 is a relationship diagram of relative energy contribution of each frequency band according to Example 2 of the present disclosure.

### DETAILED DESCRIPTION

The following embodiments are disclosed with accompanying diagrams for detailed description. For illustration clarity, many details of practice are explained in the following descriptions. However, it should be understood that these details of practice do not intend to limit the present disclosure. That is, these details of practice are not necessary in parts of embodiments of the present disclosure. Furthermore, for simplifying the drawings, some of the conventional structures and elements are shown with schematic illustrations.

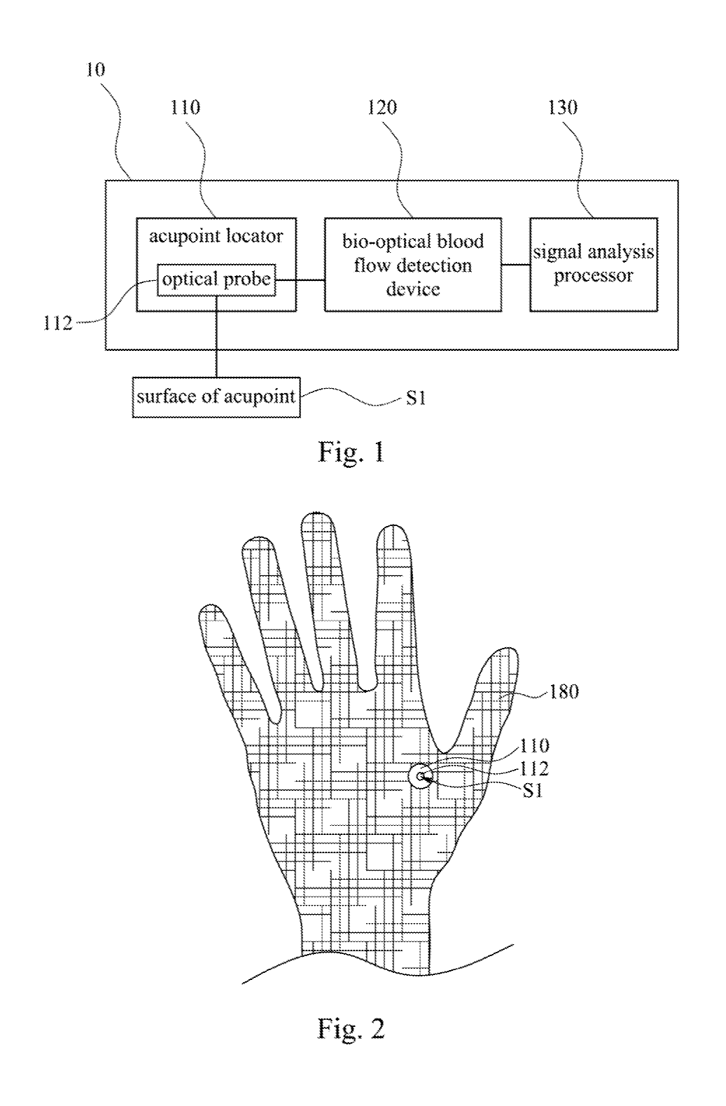

An aspect of the present disclosure provides an apparatus for detecting surface microcirculation of an acupoint. FIG. 1 is a functional module diagram of an apparatus 10 for detecting surface microcirculation of an acupoint according to one embodiment of the present disclosure. The apparatus 10 includes an acupoint locator 110, a bio-optical blood flow detection device 120 and a signal analysis processor 130.

The acupoint locator 110 includes an optical probe 112 disposed in the acupoint locator 110. The acupoint locator 110 is configured to let the optical probe 112 substantially align with and be in contact with a surface SI of the acupoint of a human subject. The acupoint may be taihsi acupoint, sanyinjiao acupoint or hegu acupoint, but not limited thereto. Preferably, the surface of the acupoint and therenear is flat, which is conductive to directly immobilize the optical probe 112 of the acupoint locator 110 on the surface S1 of the acupoint. For instance, the optical probe 112 may be pasted and thus immobilized on the surface S1 of the acupoint.

In another embodiment, the apparatus 10 further includes an elastic element configured to position the acupoint locator 110. For instance, as shown in FIG. 2, the elastic element 180 is a glove having a through opening (not marked) at a position corresponding to hegu acupoint. The through opening is configured to accommodate the acupoint locator 110. After the elastic element 180 is worn on the human subject's hand, the acupoint locator 110 may be disposed in the through opening to let the optical probe 112 directly position on the surface of hegu acupoint. In other embodiments, the shape of the elastic element and the position of the through opening may be appropriately altered based on the tested acupoint, and not limited to the embodiments exemplified above.

Referring to FIG. 1, the bio-optical blood flow detection device 120 is configured to capture microcirculation signals (e.g., microcirculation blood flow (flux) and blood volume variation) of the surface S1 of the acupoint of the human subject by the optical probe 112. The bio-optical blood flow detection device 120 may be directly connected to the optical probe 112. The bio-optical blood flow detection device 120 may be a laser Doppler flowmetry (LDF) or a photoplethysmography (PPG).

In the laser Doppler flowmetry, when laser from the optical fiber probe 112 is emitted into skin, two kinds of scattering light will be produced in the irradiated tissue. One is scattering light formed from static tissue, and frequency of the scattering light is not changed; the other is scattering light formed from dynamic tissue (e.g. erythrocytes). When light irradiates on the dynamic tissue, Doppler scattering light will be formed, and frequency thereof is changed. The scattering light may be absorbed by a light sensor and then converted to electrical signals. The electrical signals may be transferred to and calculated in the laser Doppler flowmetry. The final computing result is current blood flow variation of the measured position. The measured parameters may be blood flow flux, DC (i.e., scattering light intensity) and temperature. The blood flow flux refers to moving erythrocytes per unit volume. DC refers to a DC signal received from the formed scattering light after laser hits erythrocytes. Temperature refers to a skin surface temperature measured during the optical probe is in contact with the skin.

Characteristics of hemoglobin with oxygen are utilized in the photoplethysmography. Arterial diameter is increased, and light transmission path becomes longer, and amount of light attenuation is increased when blood pressure is increased, and thus PPG signal is reduced. Therefore, the PPG signals may be used to evaluate the state of microcirculation.

The signal analysis processor 130 is configured to receive and analyze the microcirculation signals. The processing platform of the signal analysis processor 130 may be an information processing server configured to process and analyze signals, such as servers, workstations, desktops, laptops and so on. In the embodiments that the bio-optical blood flow detection device 120 is the laser Doppler flowmetry, for frequency domain analysis, total energy of a specific frequency band is obtained by transforming microcirculation signals and accumulating energy spectral density in the specific frequency band using continuous wavelet analysis. In one embodiment, the specific frequency band is in a range of about 0.0095 Hz to about 0.021 Hz, about 0.021 Hz to about 0.052 Hz, about 0.052 Hz to about 0.145 Hz, about 0.145 Hz to about 0.6 Hz or about 0.6 Hz to about 1.6 Hz.

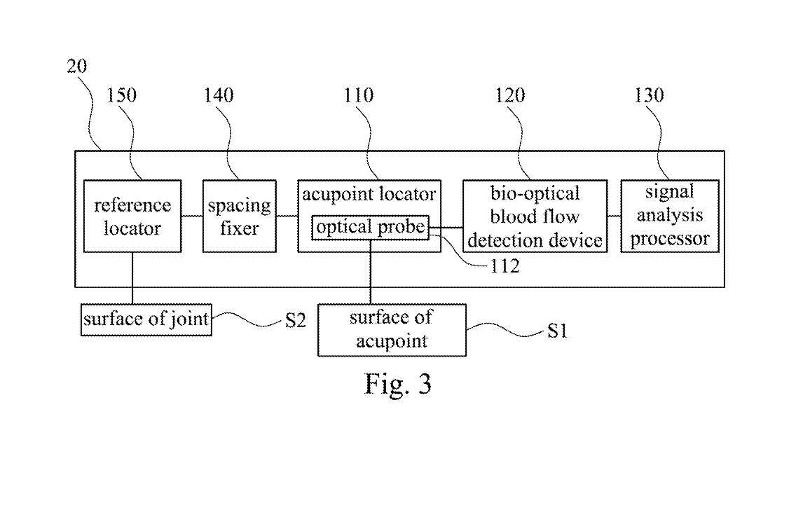

FIG. 3 is a functional module diagram of an apparatus 20 for detecting surface microcirculation of an acupoint according to another embodiment of the present disclosure. The difference between the apparatus 20 and the apparatus 10 is that the apparatus 20 further includes a spacing fixer 140 and a reference locator 150. Two opposite sides of the spacing fixer 140 are respectively connected to the reference locator 150 and the acupoint locator 110. The reference locator 150 is configured to be in contact with a surface 52 of a joint of the human subject, and the surface 52 of the joint is adjacent to the surface S1 of the acupoint. As such, the surface S2 of the joint can be acted as a reference point, and the optical probe 112 in the acupoint locator 110 can be precisely positioned on the surface S1 of the acupoint of the human subject.

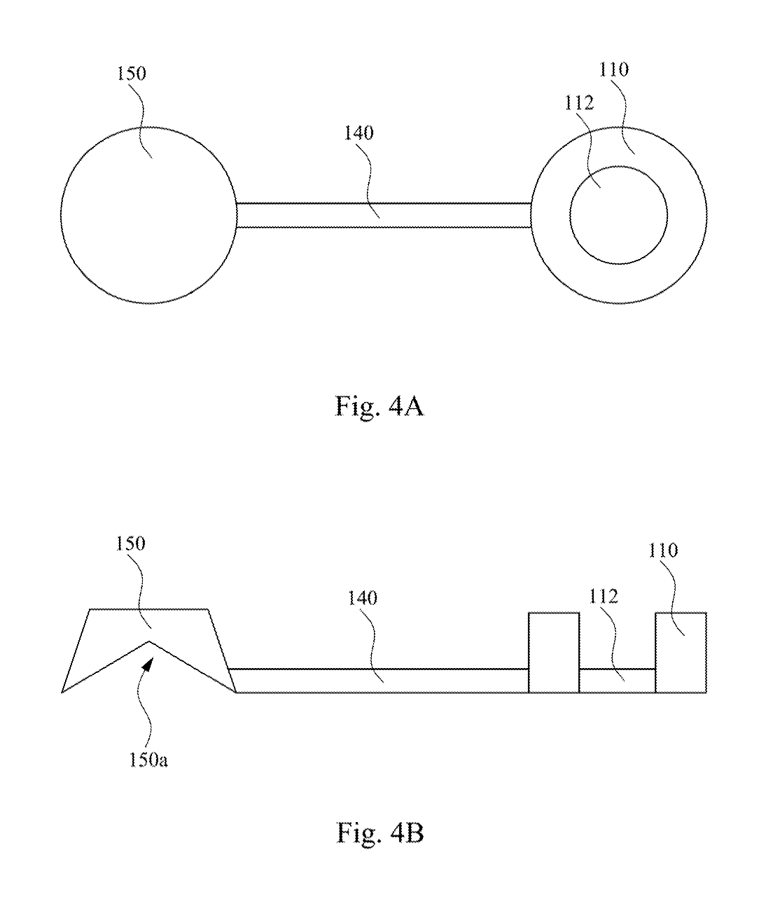

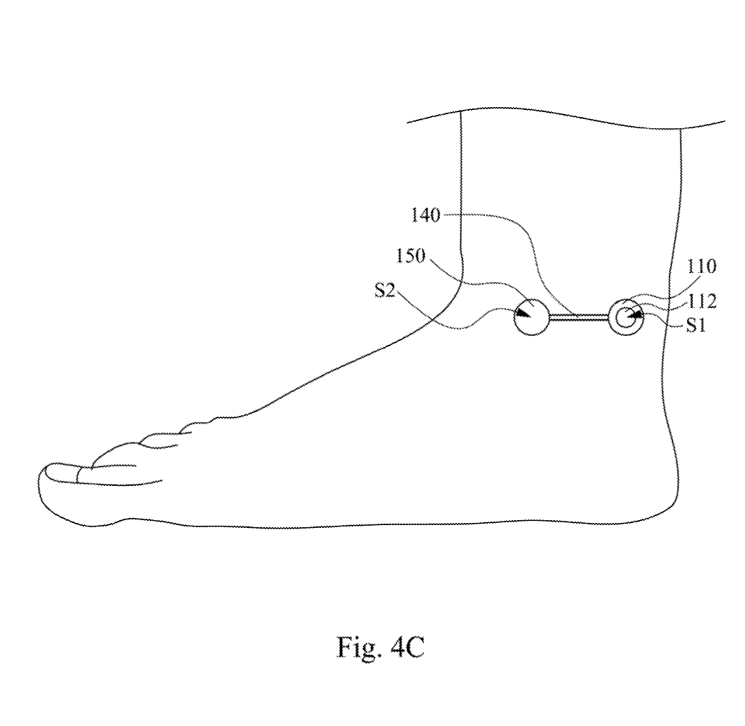

FIGS. 4A-4B respectively are top and cross-sectional views of an acupoint locator 110, a spacing fixer 140 and a reference locator 150 according to one embodiment of the present disclosure. As shown in FIGS. 4A-4B, the acupoint locator 110 has a shape of a hollow cylinder, and a through opening (not marked) is configured to accommodate the optical probe 112. The outer surface of the optical probe 112 is close to the sidewall of the opening, and thus the optical probe 112 is securely immobilized in the acupoint locator 110. The spacing fixer 140 is configured to maintain a constant spacing to ensure positioning accuracy of the surface of the acupoint. The reference locator 150 has an opening 150a configured to substantially align with and accommodate the joint. The acupoint locator 110 and the reference locator 150 may be made of elastic deformable materials. In the embodiment, the acupoint is taihsi acupoint or sanyinjiao acupoint, and the surface of the joint is internal ankle tip. For example, as shown in FIG. 4C, the reference locator 150 may accommodate and be attached to the internal ankle tip. Next, the acupoint locator 110 and the optical probe 112 may be attached to the surface of taihsi acupoint according to the spacing fixer 140. As a result, the optical probe 112 may be precisely positioned on the surface of taihsi acupoint. However, due to difference in size of human subjects, the spacings between the surface of the acupoint and the surface of the joint are also different. Therefore, a relationship of height and the spacing of human subject groups may be investigated to prepare spacing fixers 140 with various lengths. A spacing fixer 140 with an appropriate length may be selected according to the height of the subsequent human subject.

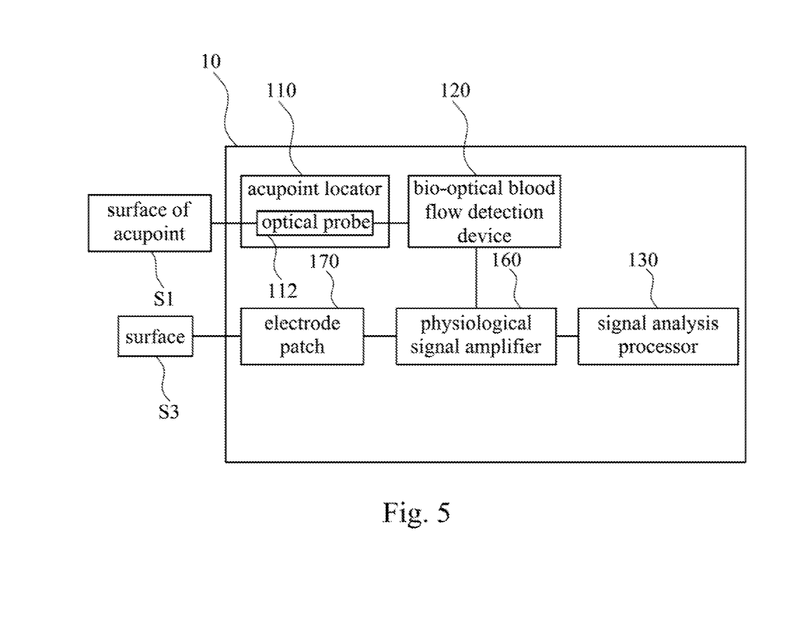

FIG. 5 is a functional module diagram of an apparatus 30 for detecting surface microcirculation of an acupoint according to further embodiment of the present disclosure. The difference between the apparatus 30 and the apparatus 10 is that the apparatus 30 further includes a physiological signal amplifier 160 and an electrode patch 170. The physiological signal amplifier 160 is configured to capture and amplify electrocardiogram (ECG) signals, and the amplified ECG signals are then transferred to the signal analysis processor 130. The ECG signal is a potential difference between inside and outside of myocardial due to weak electrical impulses formed during heartbeat. Frequency of ECG signals is in a range of 0.05 Hz to 100 Hz, and amplitude thereof is in a range of 1 mv to 10 mv, and thus the ECG signals should be amplified using the physiological signal amplifier 160. P, Q, R, S and T waves are included in one heartbeat. In one embodiment, R wave of the ECG signal is analyzed. Since the peak value of R wave is the peak value of the ECG signal, heartbeat interval obtained by R wave is less susceptible to noise interference. The electrode patch 170 is configured to paste on a surface S3 of the human subject. Specifically, the connection of the electrode patches 170 may refer to the standard Ehrlich three leads. In one specific embodiment, the electrode patches 170 are respectively pasted on right hand and left foot in accordance with the second lead. In addition, in the embodiment, the bio-optical blood flow detection device 120 is connected to the physiological signal amplifier 160 to enhance resolution of the microcirculation signals.

In the embodiment that the bio-optical blood flow detection device 120 is the laser Doppler flowmetry, the signal analysis processor 130 is configured to receive and analyze the microcirculation signals and the ECG signals to obtain a foot delay time (FST), which refers to a delay time of blood from the heart to the peripheral blood vessels. The foot delay time can be acted as an important parameter for evaluation of microcirculation. Furthermore, for time domain analysis, DCflux (i.e., average (mean) blood flow flux), DC_CV (i.e., variability of average (mean) blood flow flux), ACflux (i.e., waveform area of average (mean) blood flow), AC_CV (i.e., variability of waveform area of average (mean) blood flow), FDT_CV (i.e., variability of foot delay time), FRT (i.e., foot raising time), FRT_CV (i.e., variability of foot raising time), PW (i.e., pulse width), PW_CV (i.e., variability of pulse width) may be obtained by calculating the microcirculation signals and the ECG signals.

In other embodiments, a signal connector (not shown) and an analog to digital converter (not shown) may be disposed between the physiological signal amplifier 160 and the signal analysis processor 130. The signal connector is configured to receive the microcirculation signals and the ECG signals and then transfer those signals to the analog to digital converter. The analog to digital converter is configured to covert the microcirculation signals and the ECG signals to digital signals, and then transfer those digital signals to the signal analysis processor 130 for computing.

It is noteworthy that the apparatus of the present disclosure has excellent resolution and can be used in early detection of diseases related to abnormal blood supply of peripheral vascular bed, such as diabetes and polycystic ovary syndrome. The reason why the apparatus can be applied in early detection of diabetes and polycystic ovary syndrome will be described below in detail.

### EXAMPLES

The following Examples are provided to illustrate certain aspects of the present disclosure and to aid those of skill in the art in practicing this disclosure. These Examples are in no way to be considered to limit the scope of the disclosure in any manner.

A. Application of Early Detection of Diabetes

### Example 1

### Test Position was Taihsi Acupoint

Used apparatus were a laser Doppler flowmetry (model: moor VMS-LDF), a physiological signal amplifier, a signal connector, an analog to digital conversion card (ADC) and an electronic sphygmomanometer.

The laser Doppler flowmetry had wavelength of a light source of 400 nm to 700 nm, a measured depth of 1 mm to 2 mm, a maximum power of the light source of 6 mW, an aperture diameter of optical fiber of 0.5 mm, and instrument maximum sampling frequency of 40 Hz. Each channel corresponds to two output connectors, which can output analog signals with 0 V to 5 V. Measured parameters include blood flow, scattering light intensity and temperature.

The physiological signal amplifier was configured to capture ECG signals after giving auxiliary circuits and power supply. In the test, R wave was captured to analyze. ECG diagram was measured by using the second lead of standard Ehrlich three leads; that is, electrode patches were pasted on right hand and left foot to perform the test.

To transfer the signals to a computer, the physiological signal amplifier was connected to the signal connector through a BNC cable. Input of the signal connector had 8 channels with 37 pins, and a female-male plug of 37 pins of output was connected to the analog to digital conversion card (i.e., analog to digital converter). The shell of the signal connector was made of iron to reduce noise interference. Aluminum foil and multi-core wires were used in the signal connector to isolate external electromagnetic interference and thus to effectively keep signal strength.

The analog signals obtained during the test were converted to digital signals, and the digital signals were computed in a computer (i.e., signal analysis processor). The used analog to digital conversion card was ADLINK PCI-911DG, and input voltage was ±10V, and maximum sampling frequency was 100 kHz, and maximum resolution was 12 bits. The analog to digital conversion card supports 16 groups of analog signal input and 16 groups digital signal input/output. Meanwhile, the analog to digital conversion card provides C/C++ and LabVIEW libraries.

The used electronic sphygmomanometer was an arm-type electronic sphygmomanometer (model: MG150f, manufactured by Rossmax International Ltd.), which was used to understand a human subject whether under a stable condition before and after the test. Abnormal heartbeat or blood pressure may affect accuracy of experimental results.

In order to reduce other factors which may affect the test and to increase reliability of the test data, the human subjects should fast for 8 hours, and the environmental temperature was about 22° C., and there was no wound or bruise at the desired pasted positions. Subsequently, blood pressure and heartbeat was measured for two times with an interval of 5 minutes to check those values whether in normal ranges. A normal systolic blood pressure is in a range of 110 to 130 mmHg, and a normal diastolic blood pressure is in a range of 70 to 90 mmHg. When the difference of blood pressure between two times was in a range of ±10 mmHg, and the difference of heatbeat between two times was in a range of ±5, the optical probe of the laser Doppler flowmetry and the ECG electrode patches were pasted on the measured positions and the test was then started.

First, the optical probe of the laser Doppler flowmetry was pasted on taihsi acupoint of the human subject, and the ECG electrode patches ere pasted on right forearm and left small calf. In this operation, a reference locator was firstly immobilized on internal ankle tip of the human subject, and taihsi acupoint of the human subject was confirmed according to the position of the reference locator and a spacing fixer. The optical probe of the acupoint locator was then immobilized on the surface of taihsi acupoint. Subsequently, it was confirmed whether the microcirculation signals and ECG signals were correctly transferred to the computer through the analog to digital card. After the confirmation, continuous physiological signal acquisition was performed for 20 minutes.

The human subjects were divided into an experimental group and a control group. The human subjects of the experimental group were diabetics, and the human subjects of the control group were normal individuals (27 people). The diabetics group was further divided into A, B and C groups. A group includes 40 patients, which had a blood glucose value greater than 126 mg/dl before oral of 150 cc. 50% glucose, or had a blood glucose value greater than 200 mg/dl or HBA1C greater than or equal to 6.5% within two hours after oral of 150 cc. 50% glucose.

B group includes 30 patients, which had a blood glucose value in a range of 100-125 mg/dl before oral of 150 cc. 50% glucose, or had a blood glucose value in a range of 140-200 mg/dl or HBA1C in a range of 5.7-6.4% within two hours after oral of 150 cc. 50% glucose.

C group includes 19 patients, which had a blood glucose value lower than 100 mg/dl before oral of 150 cc. 50% glucose, or had a blood glucose value lower than 140 mg/dl or HBA1C lower than or equal to 5.6% within two hours after oral of 150 cc. 50% glucose, but suffered from diabetes complications.

The microcirculation signals captured by the laser Doppler flowmetry were converted to a relationship diagram of frequency to energy by continuous wavelet analysis. Total energy of each frequency band (FR1: about 0.0095 Hz to about 0.021 Hz; FR2: about 0.021 Hz to about 0.052 Hz; FR3: about 0.052 Hz to about 0.145 Hz; FR4: about 0.145 Hz to about 0.6 Hz; FR5: about 0.6 Hz to about 1.6 Hz) was calculated and then normalized to obtain relative energy contribution (REC).

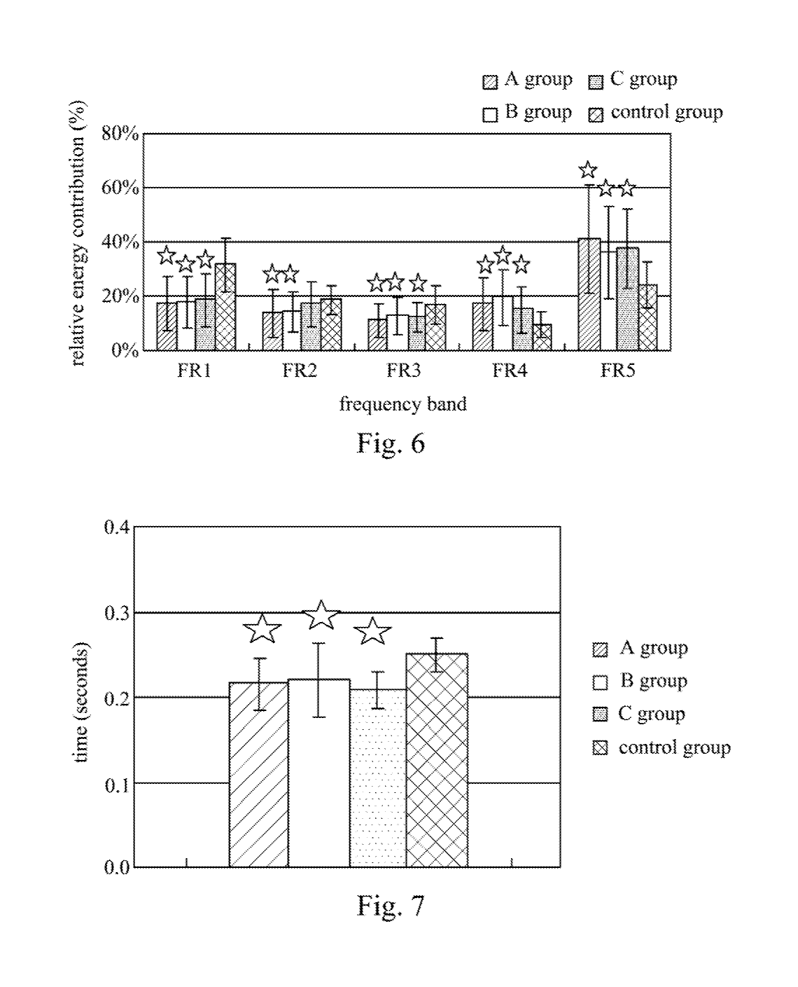

The relative energy contribution was verified by paired-t test to obtain mean value (i.e., average value) and standard deviation, and the results were shown in FIG. 6. As shown in FIG. 6, refers to p-value<0.05, which had significant difference between the experimental group and the control group. As shown in FIG. 6, in the frequency bands of FR1, FR3, FR4 and FR5, there were significant differences between A, B and C groups (i.e., experimental group) and the control group. In the frequency band of FR2, there were significant differences between A and B groups (i.e., experimental group) and the control group. The test results proved that the total energy of the specific frequency bands could be applied in early detection of diabetes.

In another aspect, a foot delay time from the heart to the peripheral blood vessels was calculated by obtaining a time difference between a lowest point of blood flow in R-R interval of the ECG signal and a previous R wave using the microcirculation signals captured by the laser Doppler flowmetry. The foot delay time was verified by paired-t test to obtain mean value and standard deviation, and the results were shown in FIG. 7. As shown in FIG. 7, ⋆ refers to p-value<0.05, which had significant difference between the experimental group and the control group. As shown in FIG. 7, there were significant differences between A, B and C groups (i.e., experimental group) and the control group. The test results proved that the foot delay time could be applied in early detection of diabetes. In practical applications, to determine whether the human subject suffers from diabetes, the total energy of the specific frequency bands and the foot delay time are confirmed to enhance accuracy of determination.

### Comparative Example 1

### Test Position was a Point Above Taihsi Acupoint and a Distance Between the Point and Taihsi Acupoint is 2 cm

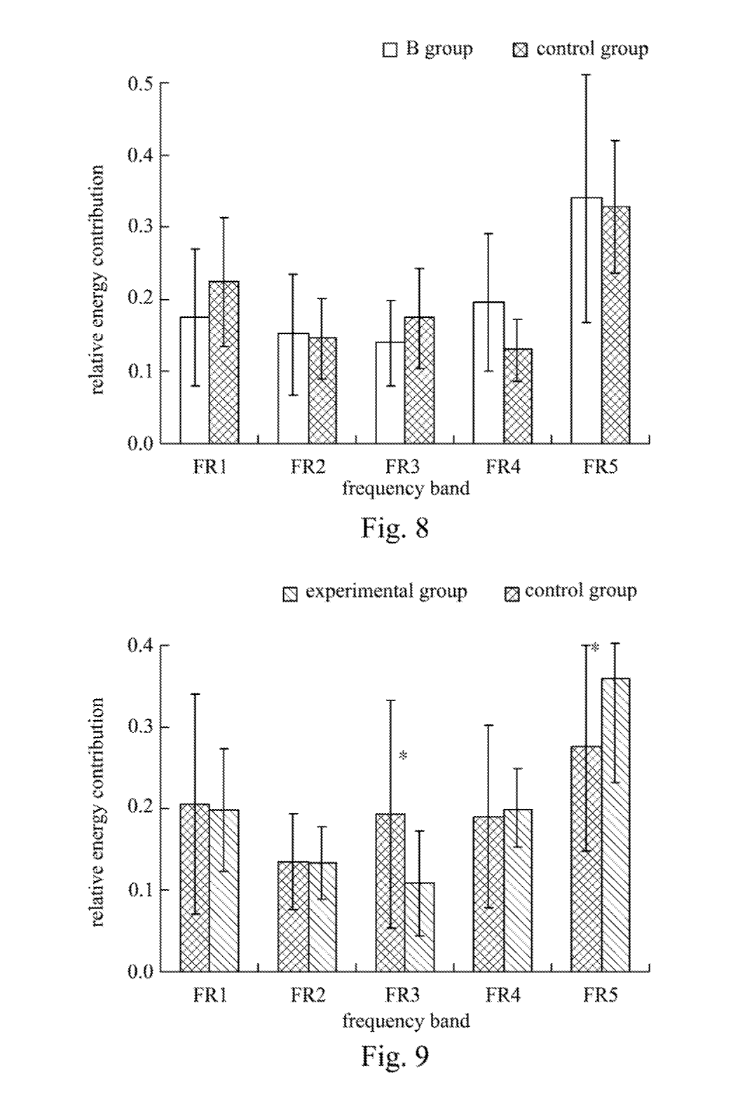

The used apparatus and analysis method was the same as those of Example 1, and thus omitted herein. The difference between Example 1 and Comparative Example 1 was that in Comparative Example 1, the test position was a surface above taihsi acupoint and a distance between the surface and taihsi acupoint is 2 cm. In Comparative Example 1, the experimental group only sampled B group. FIG. 8 is a relative energy contribution of each frequency band of B group (i.e., experimental group) and the control group. As shown in FIG. 8, there was no significance difference between B group and the control group, which mean that the testing method applied on a non-acupoint cannot determine whether the human subject suffers from the diabetes.

B. Application of Early Detection of Polycystic Ovary Syndrome

### Example 2

### Test Position was Sanyinjiao Acupoint

The used apparatus and analysis method was the same as those of Example 1, and thus omitted herein. The difference between Example 2 and Example 1 was that in Example 2, the test position was sanyinjiao acupoint. Human subjects were divided into an experimental group and a control group, and were not tested within 3 days after menstruation, and pregnant women are excluded. The human subjects of the experimental group had an amount of follicles greater than or equal to 10, or had a total area of follicles greater than or equal to 140 mm². The human subjects of the control group were non-smokers and did not suffer from obvious diseases and gynecological diseases in the past and did not take any meditation within 3 days before the test.

Similar to Example 1, the microcirculation signals captured by the laser Doppler flowmetry were converted to a relationship diagram of frequency to energy by continuous wavelet analysis. Total energy of each frequency band was calculated and then normalized to obtain relative energy contribution. The relative energy contribution was then verified by paired-t test to obtain mean value and standard deviation, and the results were shown in FIG. 9. As shown in FIG. 9, * refers to p-value<0.05, which had significant difference between the experimental group and the control group. As shown in FIG. 9, there were significant differences between the experimental group and the control group in the frequency bands of FR3 and FR5. In the frequency band of FR3, the relative energy contribution of the experimental group was significantly lower than that of the control group. In the frequency band of FR5, the relative energy contribution of the experimental group was significantly higher than that of the control group. The test results proved that the frequency bands of FR3 and FR5 could be applied in early detection of polycystic ovary syndrome.

Given the above, the apparatus can be used for early detection of diabetes, and the detection acupoint is taihsi acupoint, and the specific frequency bands preferably are FR1, FR3, FR4 and FR5, and even better are FR1 and FR5. The foot delay time may also be applied in early detection of diabetes. In another aspect, the apparatus may be used for early detection of polycystic ovary syndrome, and the detection acupoint is sanyinjiao acupoint, and the specific frequency bands preferably are FR3 and FR5.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fail within the scope of the following claims.

What is claimed is:

1. An apparatus for detecting surface microcirculation of an acupoint, comprising:
an acupoint locator including an optical probe disposed in the acupoint locator, and the acupoint locator configured to let the optical probe substantially align and be in contact with a surface of the acupoint of a human subject;
a bio-optical blood flow detection device configured to capture microcirculation signals of the surface of the acupoint of the human subject by the optical probe, wherein the bio-optical blood flow detection device is a laser Doppler flowmetry (LDF) or a photoplethysmography (PPG); and
a signal analysis processor configured to receive and analyze the microcirculation signals.

2. The apparatus of claim 1, further comprising:
a reference locator configured to be in contact with a surface of a joint of the human subject, and the joint adjacent to the acupoint; and
a spacing fixer, and two opposite sides of the spacing fixer respectively connected to the reference locator and the acupoint locator.

3. The apparatus of claim 2, wherein the acupoint is taihsi acupoint or sanyinjiao acupoint, and the surface of the joint is internal ankle tip.

4. The apparatus of claim 3, wherein the reference locator has an opening configured to substantially align with and accommodate the internal ankle tip.

5. The apparatus of claim 1, wherein the bio-optical blood flow detection device is the laser Doppler flowmetry, and the signal analysis processor is configured to transform the microcirculation signals to total energy of a specific frequency band, and the specific frequency band is in a range of about 0.0095 Hz to about 0.021 Hz, about 0.021 Hz to about 0.052 Hz, about 0.052 Hz to about 0.145 Hz, about 0.145 Hz to about 0.6 Hz or about 0.6 Hz to about 1.6 Hz.

6. The apparatus of claim 1, wherein the acupoint is taihsi acupoint, and the apparatus is configured for early detection of diabetes.

7. The apparatus of claim 1, wherein the acupoint is sanyinjiao acupoint, and the apparatus is configured for early detection of polycystic ovary syndrome.

8. The apparatus of claim 1, further comprising:
an electrode patch configured to paste on a surface of the human subject; and
a physiological signal amplifier configured to capture and amplify electrocardiogram (ECG) signals through the electrode patch.

9. The apparatus of claim 8, wherein the bio-optical blood flow detection device is the laser Doppler flowmetry, and the signal analysis processor is further configured to receive and analyze the electrocardiogram signals to obtain a foot delay time (FDT).

10. The apparatus of claim 1, wherein the acupoint is hegu acupoint.

11. The apparatus of claim 1, further comprising an elastic element configured to position the acupoint locator.
